(19) United States
(12) Patent Application Publication
(10) Pub. No.: US 2015/0017706 A1
(43) Pub. Date: Jan. 15, 2015

(54) CAPTURED CARBON DIOXIDE FOR ALGACULTURE

(71) Applicant: Carbon Engineering Limited Partnership, Calgary (CA)

(72) Inventors: Arvinder Pal Singh Kainth, Calgary (CA); Kenton Robert Heidel, Calgary (CA); Matthew Alex Henderson, Calgary (CA); Geoffrey James Holmes, Calgary (CA); Jane Anne Ritchie, Calgary (CA); David William Keith, Cambridge, MA (US)

(21) Appl. No.: 14/377,485

(22) PCT Filed: Feb. 8, 2013
(86) PCT No.: PCT/US2013/025444

§ 371 Date: Aug. 7, 2014

Related U.S. Application Data
(60) Provisional application No. 61/596,983, filed on Feb. 9, 2012.

(51) Int. Cl. C12N 1/12; C12M 1/34; C12M 1/00

(52) U.S. Cl. CPC C12N 1/12; C12M 45/06; C12M 29/04; C12M 41/32; C12M 21/02
USPC 435/257.1; 435/289.1; 435/297.1

(57) ABSTRACT

Enhancing growth of algae in an algaculture facility includes contacting a growth medium with a gas including carbon dioxide, transferring some of the carbon dioxide to the growth medium to yield an enriched growth medium, and providing the enriched growth medium to the algaculture facility. The concentration of dissolved carbon dioxide in the enriched growth medium exceeds the concentration of dissolved carbon dioxide in the growth medium, where dissolved carbon dioxide includes ions formed by the reaction of carbon dioxide with a species in solution. The growth medium may be obtained from the algaculture facility, and may be filtered or otherwise processed before or after contacting the growth medium with the gas. Providing the enriched growth medium to the algaculture facility increases the concentration of dissolved carbon dioxide in the bulk growth medium of the algaculture facility.

### CROSS-REFERENCE TO RELATED APPLICATION

This application claims priority to U.S. Application Ser. No. 61/596,983, filed on Feb. 9, 2012, which is incorporated by reference herein in its entirety.

### TECHNICAL FIELD

This invention is related to the capture of carbon dioxide and the delivery of the captured carbon dioxide to an algaculture facility.

### BACKGROUND

Algal cultivation systems have been used in the production of foodstuffs, food additives, fertilizers, bioplastics, chemical feedstocks, pharmaceuticals, and algal fuels (e.g., straight vegetable oil, biodiesel, aviation biofuel, bioethanol, biogasoline, biomethanol, biobutanol, and other biofuels). Examples of algal cultivation systems include open ponds and photobioreactors (e.g., closed systems which incorporate a source of light), in which algae is cultivated for use (e.g., for the production of biofuel).

Carbon dioxide has been supplied to algal cultivation systems to promote growth of the algae. For example, gaseous carbon dioxide has been bubbled directly into algal cultivation systems. This process can be inefficient, however, since the gaseous carbon dioxide tends to bubble out of solution and into the atmosphere above the algal cultivation system before it is consumed by the algae.

### SUMMARY

As described herein, a carbon dioxide capture facility is coupled to an algaculture facility to enhance growth of algae in the algaculture facility. As used herein, “carbon dioxide capture facility” generally refers to a system or apparatus configured to transfer carbon dioxide from a gas to an aqueous solution. “Algaculture facility” generally refers to a system including a reservoir with a growth medium in which algae are cultivated and from which the algae, a consumer of the algae, or a metabolic product or derivative thereof is harvested. The reservoir may be open or enclosed. Examples of reservoirs include an open pond, a photobioreactor, a trickle filter, a closed pond, a greenhouse pond, or a combination or modification thereof. The growth medium is an aqueous solution or heterogeneous mixture including dissolved gases, organic and inorganic compounds, particulate matter, living organisms (e.g., algae), and the like. The algae may be single- or multi-cellular, naturally occurring or genetically modified algae.

In one aspect, enhancing growth of algae in an algaculture facility includes contacting a growth medium with a gas comprising carbon dioxide, transferring some of the carbon dioxide from the gas to the growth medium to yield an enriched growth medium, and providing the enriched growth medium to an algaculture facility comprising algae, thereby enhancing growth of the algae in the algaculture facility. The concentration of dissolved carbon dioxide in the enriched growth medium exceeds the concentration of dissolved carbon dioxide in the growth medium, where dissolved carbon dioxide includes ions formed by the reaction of carbon dioxide with a species in solution, including such ions formed in the growth medium and added to the growth medium.

Implementations may include one or more of the following features. For example, the ions formed by the reaction of carbon dioxide with a species in solution can be provided to the algaculture facility in the form of a solution, a slurry, or a salt. Species that react with carbon dioxide in solution include, for example, water, carbonate, dibasic phosphate, and tribasic phosphate.

In some cases, the growth medium is a portion of the bulk growth medium in the algaculture facility. The growth medium may be obtained or removed from the bulk growth medium before it is contacted with the gas. The process of removing the growth medium from the algaculture facility, transferring some of the carbon dioxide to the growth medium to yield an enriched growth medium, and providing the enriched growth medium to the algaculture facility may be a batch process or a continuous process. Algae in the algaculture facility, a consumer thereof, a metabolic product thereof, or a derivative of a metabolic product thereof may be harvested from the algaculture facility (e.g., after providing the enriched growth medium to the algaculture facility).

The enriched growth medium may be subjected to a filtration, dialysis, reverse osmosis, or ion exchange process to alter the concentration of dissolved carbon dioxide in the enriched growth medium before providing the enriched growth medium to the algaculture facility. In some cases, the dissolved carbon dioxide is crystallized from the enriched growth medium to yield a solid carbon dioxide-containing salt, and the solid carbon dioxide-containing salt is provided to the algaculture facility. The enriched growth medium may include a buffer species, and the buffer species may be crystallized from the enriched growth medium to yield a solid buffer species. In some cases, the solid buffer species is provided to or conveyed to the carbon dioxide capture facility, and the enriched growth medium is provided to the algaculture facility.

In some implementations, dissolved compounds or particulate matter is removed (e.g., by filtering) from the growth medium before contacting the growth medium with the gas in the carbon dioxide capture facility. The dissolved compounds or the particulate matter may be provided (e.g., returned) to the algaculture facility. Water may be removed from the growth medium before contacting the growth medium with the gas. The water (or water from another source) may be added to the enriched growth medium before providing the enriched growth medium to the algaculture facility. In some cases, the growth medium includes an additive (e.g., a catalyst) that increases the rate of transfer of the carbon dioxide from the gas to the growth medium. In certain cases, the growth medium is exposed to light while contacting the growth medium with the gas.

In another aspect, a system includes an algaculture facility including a growth medium, a carbon dioxide capture facility coupled to the algaculture facility, and a pump configured to transfer a portion of the growth medium from the algaculture facility to the carbon dioxide capture facility. The carbon dioxide capture facility is configured to transfer carbon dioxide from a gas to the portion of the growth medium to yield an enriched growth medium, such that the concentration of dissolved carbon dioxide in the enriched growth medium exceeds the concentration of dissolved carbon dioxide in the growth medium, where dissolved carbon dioxide includes ions formed by the reaction of carbon dioxide with a species in solution, including such ions formed in the growth medium and added to the growth medium.

Implementations may include one or more of the following features. For example, the carbon dioxide capture facility may be operatively or fluidically coupled to the algaculture facility. The system may include a second pump configured to transfer the enriched growth medium to the algaculture facility. In some cases, the system includes a first separation unit and/or a second separation unit fluidically interposed between the algaculture facility and the carbon dioxide capture facility. The first separation unit may be configured to remove one or more components from the growth medium before the growth medium is transferred to the carbon dioxide capture facility, or to remove one or more components from the enriched growth medium before the enriched growth medium is transferred to the algaculture facility. In addition, or alternatively, the second separation unit may be configured to increase or decrease a concentration of one or more components of the growth medium before the growth medium is transferred to the carbon dioxide capture facility, or to increase or decrease a concentration of one or more components of the enriched growth medium before the enriched growth medium is transferred to the algaculture facility.

Increasing the concentration of dissolved carbon dioxide in the algaculture facility enhances growth of the algae. Moreover, providing the carbon dioxide as a dissolved component in solution reduces loss of carbon dioxide to the atmosphere associated with providing gaseous carbon dioxide directly to the growth medium.

### BRIEF DESCRIPTION OF THE DRAWINGS

FIG. 1 depicts a system including a carbon dioxide capture facility coupled to an algaculture facility.

FIG. 2 depicts a system including a carbon dioxide capture facility coupled to an algaculture facility, with a separation unit functionally interposed between the carbon dioxide capture facility and the algaculture facility.

FIG. 3 depicts a system including a carbon dioxide capture facility coupled to an algaculture facility, with a processing unit functionally interposed between the carbon dioxide capture facility and the algaculture facility.

FIG. 4 depicts a carbon dioxide capture facility coupled to an algaculture facility, with a concentration unit and a dilution unit functionally interposed between the carbon dioxide capture facility and the algaculture facility.

FIG. 5 is a flowchart showing a process including capture of carbon dioxide and delivery of the captured carbon dioxide to an algaculture facility.

### DETAILED DESCRIPTION

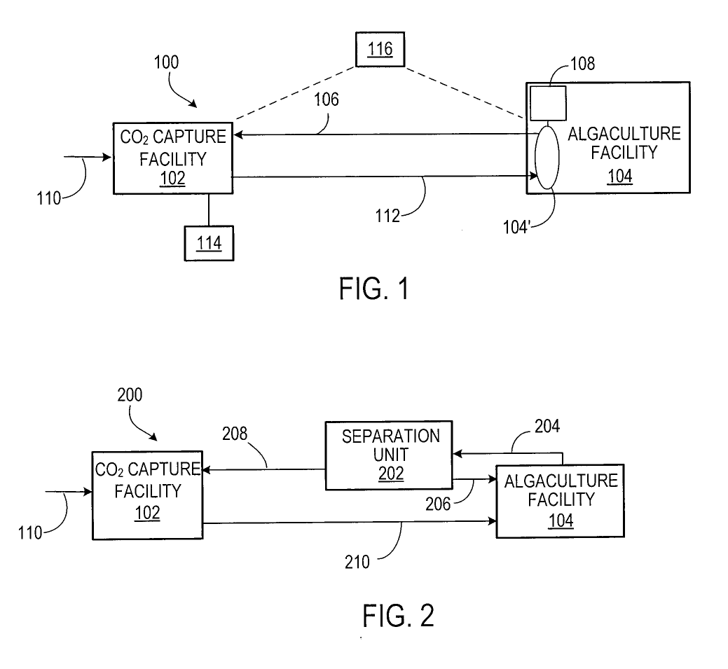

Referring to FIG. 1, system 100 includes carbon dioxide capture facility 102 coupled to algaculture facility 104. Algaculture facility 104 includes reservoir 104′. Reservoir 104′ includes algae in a bulk growth medium. Carbon dioxide capture facility 102 may be fluidically coupled to algaculture facility 104. As used herein, “carbon dioxide capture facility” generally refers to a system or apparatus configured to transfer carbon dioxide from a gas to a liquid. The gas may be a gaseous mixture, such as air. The liquid is typically an aqueous solution. “Algaculture facility” generally refers to a system including a reservoir with a growth medium in which algae are cultivated and from which at least one of: the algae, a consumer of the algae, or a metabolic product or derivative thereof is harvested. The reservoir may be open or enclosed. Examples of reservoirs include an open pond, a photobioreactor, a trickle filter, a closed pond, a greenhouse pond, or a combination or modification thereof. The growth medium is an aqueous solution or heterogeneous mixture including dissolved gases, organic and inorganic compounds, particulate matter, living organisms (e.g., algae), and the like. The algae may be single- or multi-cellular, naturally occurring or genetically modified algae. A consumer of the algae may include vertebrate or invertebrate organisms such as fish, frogs, insects, and the like. Algal metabolic products include lipids, fatty acids, and polysaccharides such as starch. Derivatives of these products include, for example, ethanol, butanol, biodiesel, and isopentenol.

As depicted in FIG. 1, a portion of growth medium 106 from bulk growth medium in algaculture facility 104 is provided to carbon dioxide capture facility 102. Growth medium 106 is an aqueous mixture including dissolved gases, organic and inorganic compounds, particulate matter, algae, and the like from reservoir 104′ of algaculture facility 104. Pump 108 facilitates transfer of growth medium 106 to carbon dioxide capture facility 102. In carbon dioxide capture facility 102, carbon dioxide is transferred from gas 110 (e.g., ambient air, industrial flue gas, or the like) to growth medium 106, dissolving in and reacting with species in the growth medium to yield enriched growth medium 112. The concentration of dissolved carbon dioxide in enriched growth medium 112 exceeds the concentration of dissolved carbon dioxide in growth medium 106. As used herein, “dissolved carbon dioxide” includes carbon dioxide in solution and ions formed by the reaction of carbon dioxide with species in solution, including such ions formed in the growth medium and added to the growth medium (e.g., in the form of a solution, salt, slurry, or the like). Species that react with carbon dioxide in solution include, for example, water, carbonate, dibasic phosphate, tribasic phosphate, amino acids, alkanolamines, and the like, or reaction products thereof.

The transfer of carbon dioxide from gas 110 to growth medium 106 may be achieved, for example, by blowing gas 110 through a structured packing material such as XF12560 Cross Fluted Film Fill Media available from Brentwood Industries (Reading, Pa.). The packing material is wetted by the growth medium 106 from algaculture facility 104. The direction of gas flow with respect to the direction of the flow of growth medium 106 over the packing material may be counter flow, with the growth medium flowing downward through the packing material and gas 110 blown upwards. The arrangement may be co-current with the direction of gas flow and growth medium both flowing in the downwards direction. The arrangement may also be cross flow, in which growth medium 106 flows downward through the packing material and gas 110 is blown horizontally through the packing material, as described in U.S. Patent Publication No. 2010/0064890 entitled “CARBON DIOXIDE CAPTURE METHOD AND FACILITY,” which is incorporated herein by reference. Gas 110 can be forced to flow at a selected velocity into carbon dioxide capture facility 102 (e.g., in a range between 0.1 m/s and 10 m/s, or between 0.5 m/s and 2 m/s). In some cases, carbon dioxide capture facility 102 is configured such that gas 110 is bubbled into growth medium 106.

In one example, formation of dissolved carbon dioxide (e.g., HCO₃⁻) in an aqueous growth medium including carbonate ions can occur via the reaction of carbonate ions with water to yield bicarbonate and the reaction of carbon dioxide with hydroxide to yield bicarbonate, as shown below:

CO₃²⁻+H₂O→OH⁻+HCO₃⁻ (1)

and

OH⁺+CO₂→HCO₃⁻ (2)

resulting in the net reaction:

CO₃²⁻+CO₂+H₂O→2HCO₃⁻. (3)

Formation of dissolved carbon dioxide (e.g., HCO₃⁻) in growth medium including carbonate ions can also occur via the reaction of carbon dioxide with water to yield carbonic acid and the deprotonation of carbonic acid to yield bicarbonate, as shown below:

CO₂+H₂O→H₂CO₃ (4)

and

H₂CO₃→H⁺+HCO₃⁻, (5)

with the carbonate ions reacting with the generated proton:

H⁺+CO₃²→HCO₃⁻. (6)

and resulting in the net reaction:

CO₃²⁻+CO₂+H₂O→2HCO₃⁻. (3)

These mechanisms, in which carbon dioxide reacts with hydroxide, as in reaction (2), and with water, as in reaction (4), may occur simultaneously.

In other examples, carbon dioxide in the gas reacts with an amino acid species (e.g., potassium argenate, potassium taurate, etc.), an alkanolamine species (e.g., monoethanolamine, diethanolamine, methyl diethanolamine, etc.), or the like to yield dissolved carbon dioxide.

As described with respect to an aqueous solution in U.S. patent application Ser. No. 13/606,926 entitled “TARGET GAS CAPTURE” and filed on Sep. 7, 2012, which is incorporated by reference herein, buffer species in the growth medium react with protons formed during the absorption of carbon dioxide by the growth medium (e.g., protons generated as shown by reaction (5) are consumed in reaction (6), driving reaction (5) to the right), thereby increasing the concentration of bicarbonate ions in solution (i.e., increasing the concentration of dissolved carbon dioxide).

Additives may be combined with growth medium 106 in any portion of system 100 (e.g., carbon dioxide capture facility 102 and/or algaculture facility 104). Additives may include, for example, water, buffer species, catalysts, and additives generally known to be used in algal cultivation.

Growth medium 106 processed in carbon dioxide capture facility 104 may include an additive such as a catalyst that enhances the rate of carbon dioxide transfer from gas 110 to the growth medium (e.g., enhances the rate of reaction (3) above). In one example, the catalyst is added to growth medium 106 in carbon dioxide capture facility 102. In general, the catalyst increases the rate of reaction between carbon dioxide and a species in the growth medium (e.g., water or hydroxide), thereby enhancing the transfer of carbon dioxide from gas 110 to growth medium 106. Examples of suitable catalysts include sodium or potassium hypochlorite, sodium or potassium hypobromite, sodium or potassium arsenite, zinc triazacyclododecane, zinc tetraazacyclododecane, and naturally occurring and genetically modified forms of carbonic anhydrase. In some cases, catalyst concentration in growth medium 106 may range from 0.1 gram per liter of catalyst to 100 grams per liter of catalyst, with molar concentrations dependent on the molar mass of the chosen catalyst.

Referring again to FIG. 1, enriched growth medium 112 is provided to algaculture facility 104. The concentration of dissolved carbon dioxide in enriched growth medium 112 exceeds the concentration of dissolved carbon dioxide in growth medium 106. Pump 114 facilitates transfer of enriched growth medium 112 from carbon dioxide capture facility 102 to algaculture facility 104. Enriched growth medium 112 is combined with the bulk of the growth medium in algaculture facility 104 from which growth medium 106 was removed, thereby increasing the concentration of dissolved carbon dioxide in the bulk growth medium in the algaculture facility. Increasing the concentration of dissolved carbon dioxide in the bulk of growth medium in algaculture facility 104 by the addition of enriched growth medium 112 to the bulk of growth medium in the algaculture facility increases the availability of dissolved carbon dioxide for algae in the algaculture facility, thereby enhancing growth of the algae. The algae in the bulk growth medium can consume dissolved carbon dioxide in its molecular form as aqueous carbon dioxide (CO_{2(aq)} or as the bicarbonate ion (HCO₃⁻). Moreover, providing dissolved carbon dioxide reduces the loss of carbon dioxide to the atmosphere associated with providing gaseous carbon dioxide directly to the algaculture facility.

As depicted in FIG. 1, system 100 can be a continuous or batch system. In a continuous system, after initial start-up, growth medium 106 flows to carbon dioxide capture facility 102 and enriched growth medium 112 flows to algaculture facility 104 simultaneously.

In some embodiments, one or more components of system 100 (e.g., components of carbon dioxide capture facility 102, algaculture facility 104, or both) are constructed of or include transparent components such that the algae are exposed to natural or artificial light, thereby promoting photosynthesis and thus growth of the algae. In one example, system 100 includes light pipe technology to supply light to the algae.

In some cases, system 100 includes control system 116 operatively coupled to carbon dioxide capture facility 102, algaculture facility 104, or both. Control system 116 may be a controller or multiple controllers (e.g., in a master-slave arrangement), and may include one or more processors and memory units. A memory unit may store instructions to control components of carbon dioxide capture facility 102, algaculture facility 104, or both. (e.g., user interface, valves, pumps, test equipment to assess dissolved carbon dioxide concentration, etc.).

Referring to FIG. 2, system 200 includes carbon dioxide capture facility 102 coupled to algaculture facility 104. Separation unit 202 is functionally interposed between algaculture facility 104 and carbon dioxide capture facility 102. Some or all of the features and components of system 100 shown or described with respect to FIG. 1 may be incorporated in system 200. For example, system 200 may include one or more of pumps 108 and 114 and controller system 116.

A portion of growth medium 204 is removed from the algaculture facility 104 and supplied to separation unit 202. Separation unit 202 may include one or more of a nanofiltration system, a micro filtration system, an ultrafiltration system, a reverse osmosis system, an electrodialysis system, a diffusion dialysis system, a settling tank, or a similar system in which components are separated from growth medium 204 based on size, mass, ionic charge, hydrodynamic radius, or other characteristic.

Separation unit 202 separates components 206 (e.g., algae and components that promote algae growth such as nutrients in the form of dissolved or particulate matter, etc.) from growth medium 204 and returns these components to algaculture facility 104, while species that promote carbon dioxide capture (e.g., carbonate buffer species, phosphate buffer species, amino acid based buffer species, or other buffer species) are provided via modified growth medium 208 to carbon dioxide capture facility 102.

Carbon dioxide from gas 110 is transferred to modified growth medium 208 in carbon dioxide capture facility 102 to yield enriched growth medium 210 with a concentration of dissolved carbon dioxide exceeding that of growth medium 204 and modified growth medium 208. Enriched growth medium 210 is provided to the bulk growth medium in algaculture facility 104.

Additives may be combined with growth medium 204 in any portion of system 200 (e.g., carbon dioxide capture facility 102, algaculture facility 104, and/or separation unit 202). Additives may include, for example, water, buffer species, catalysts, and additives generally known to be used in algal cultivation.

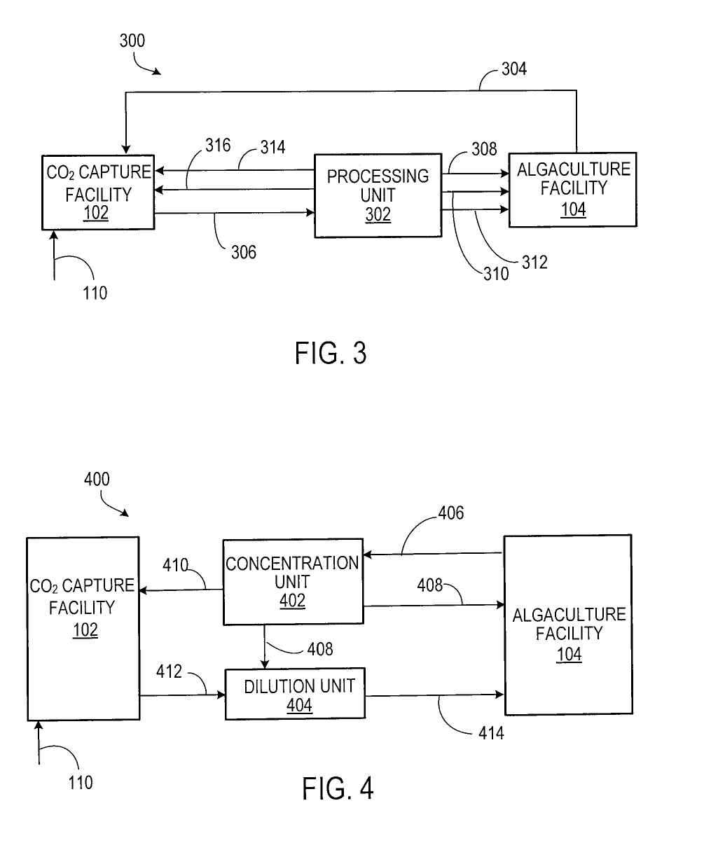

Referring to FIG. 3, system 300 includes carbon dioxide capture facility 102 coupled to algaculture facility 104. Processing unit 302 is functionally interposed between carbon dioxide capture facility 102 and algaculture facility 104. Some or all of the features of system 100 shown or described with respect to FIG. 1 and system 200 shown or described with respect to FIG. 2 may be incorporated in system 300. For example, system 300 may include pumps 108 and 114 and/or control system 116 shown in FIG. 1, separation unit 202 shown in FIG. 2, or a combination thereof.

Growth medium 304 from algaculture facility 104 is provided to carbon dioxide capture facility 102. In carbon dioxide capture facility 102, carbon dioxide is transferred from gas 110 to growth medium 304 as dissolved carbon dioxide. Enriched growth medium 306 is provided to processing unit 302. The concentration of dissolved carbon dioxide in enriched growth medium 306 exceeds the concentration of dissolved carbon dioxide in growth medium 304.

In one example, processing unit 302 is a separation unit, such as a membrane separation unit. The membrane separation unit may be one or more of, for example, a nanofiltration unit, a reverse osmosis unit, an electrodialysis unit, a diffusion dialysis unit, or other filtration or ion exchange unit configured to remove dissolved compounds, suspended solids, or particulate matter (e.g., catalysts, enzymes, and the like) from enriched growth medium 306. In some cases, processing unit 302 separates at least some of a buffer species from enriched growth medium 306 before the enriched growth medium is provided to algaculture facility 104. Processing unit 302 yields modified enriched growth medium 308 having a concentration of dissolved carbon dioxide exceeding that of enriched growth medium 306.

Separation unit 302 may operate on enriched growth medium 306 by decreasing the ratio of dissolved carbon dioxide to buffer species. For example, if enriched growth medium 306 includes approximately 0.2 moles per liter of potassium carbonate and 0.1 moles per liter of potassium bicarbonate, then enriched growth medium 306 would include approximately 0.9 moles per liter of potassium carbonate and 0.1 moles per liter of potassium bicarbonate, and modified enriched growth medium 312 would include 0.125 moles per liter of potassium carbonate and 0.25 moles per liter of potassium bicarbonate. Alternatively, if growth medium 306 includes approximately 0.008 moles per liter of sodium carbonate and 0.035 moles per liter of sodium bicarbonate, then enriched growth medium 306 would include 0.48 moles per liter of sodium carbonate and 0.05 moles per liter of sodium bicarbonate, and stream 312 would include 0.01 moles per liter of sodium carbonate and 0.06 moles per liter of sodium bicarbonate.

In another example, processing unit 302 is a crystallization unit that reduces the solubility of the dissolved carbon dioxide in enriched growth medium 306 such that the dissolved carbon dioxide precipitates from the enriched growth medium as a solid carbon dioxide-containing salt such as sodium bicarbonate or potassium bicarbonate. The solid carbon dioxide-containing salt is provided to the algaculture facility 104 as a slurry or solid via stream or conveyor 310. As used herein, “conveyor” generally refers to an apparatus, such as an auger, a pneumatic conveyor, or a belt conveyor, capable of transporting a solid or a slurry. The carbon dioxide-containing salt conveyed to algaculture facility 104 dissolves in the bulk growth medium and supplies carbon dioxide to the algae in a form such as bicarbonate ions or aqueous carbon dioxide.

In another example, processing unit 302 is a crystallization unit that precipitates a buffer species present in enriched growth medium 306 to form a solid buffer species (e.g., sodium carbonate). The solid buffer species can be returned to carbon dioxide capture facility 102 via stream or conveyor 314. Modified enriched growth medium 312, provided to algaculture facility 104, has approximately the same concentration of dissolved carbon dioxide as enriched growth medium 306, but with a higher ratio of dissolved carbon dioxide to buffer species.

In some cases, processing unit 302 is a membrane distillation unit or an evaporator that separates water from enriched growth medium 306 as water vapor until the solubility limit of the dissolved carbon dioxide or the buffer species is reached and precipitation of either component occurs from the enriched growth medium.

In certain cases, processing unit 302 is a chiller that reduces the temperature of enriched growth medium 306 until the solubility limit of the dissolved carbon dioxide or the buffer species is reached and precipitation occurs. In one example, when dissolved carbon dioxide is in solution as potassium bicarbonate, precipitation may be induced by removing water from enriched growth medium 306 or by chilling the enriched growth medium to reach the solubility limit of potassium bicarbonate, which then precipitates from solution. Solid potassium bicarbonate may be provided to algaculture facility 104 via stream or conveyor 310 as a source of carbon dioxide for algae.

Processed growth medium 316 from processing unit 302 may be cycled back through carbon dioxide capture facility 102 for additional carbon dioxide capture. Processed growth medium 316 may have a lower concentration of dissolved carbon dioxide than enriched growth medium 306.

Additives may be combined with growth medium 306 in any portion of system 300 (e.g., carbon dioxide capture facility 102, algaculture facility 104, and/or processing unit 302). Additives may include, for example, water, buffer species, catalysts, and additives generally known to be used in algal cultivation.

Referring to FIG. 4, system 400 includes carbon dioxide capture facility 102 and algaculture facility 104. Concentration unit 402 and dilution unit 404 are functionally interposed between carbon dioxide capture facility 102 and algaculture facility 104. Some or all of the features of system 100 shown or described with respect to FIG. 1, system 200 shown or described with respect to FIG. 2, and system 300 shown or described with respect to FIG. 3 may be incorporated in system 400. For example, system 400 may include pumps 108 and 114 and/or control system 116 shown in FIG. 1, separation unit 202 shown in FIG. 2, processing unit 302 shown in FIG. 3, or a combination thereof.

Growth medium 406 from algaculture facility 104 is provided to concentration unit 402. Concentration unit 402 may be, for example, a reverse osmosis unit, nanofiltration unit, ultrafiltration unit, microfiltration unit, membrane distillation unit, distillation unit, an evaporator, or another similar system that separates water from growth medium 406 based on solubility, mass, size, hydrophobicity, hydrodynamic radius, or ionic charge, or boiling point. Concentration unit 402 separates a portion of water 408 from growth medium 406 to yield modified growth medium 410 and water. Modified growth medium 410 is provided to carbon dioxide capture facility 102. Water 408 is provided to dilution unit 404, returned to algaculture facility 104, or both.

In carbon dioxide capture facility 102, gas 110 is contacted with modified growth medium 410 from concentration unit 402 to transfer carbon dioxide from the gas to the modified growth medium. Enriched growth medium 412, with a concentration of dissolved carbon dioxide exceeding that of modified growth medium 410, is provided to dilution unit 404. Enriched growth medium 412 can be combined with water 408, or other source of water, in dilution unit 404 to yield modified enriched growth medium 414. Modified enriched growth medium 414, with a concentration of dissolved carbon dioxide exceeding that of growth medium 406, is provided to algaculture facility 104, thereby increasing the concentration of dissolved carbon dioxide in the bulk growth medium in the algaculture facility.

Additives may be combined with growth medium 406 in any portion of system 400 (e.g., carbon dioxide capture facility 102, algaculture facility 104, concentration unit 402 and/or dilution unit 404). Additives may include, for example, water, buffer species, catalysts, and additives generally known to be used in algal cultivation.

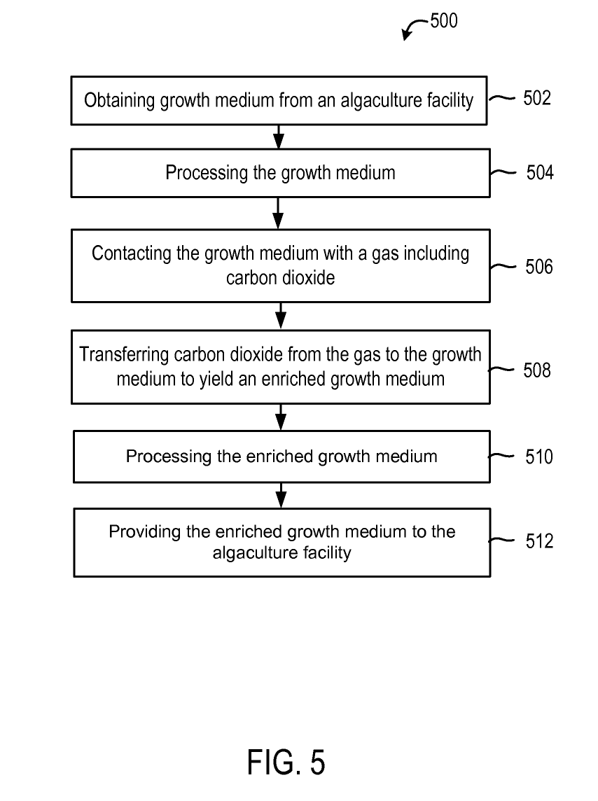

FIG. 5 shows features of process 500 for enhancing growth of algae in an algaculture facility. In 502, growth medium is obtained from an algaculture facility. The growth medium may be a portion of the bulk growth medium in the algaculture facility. In 504, the growth medium is processed to increase or decrease the concentration of a component in the growth medium. Processing may include filtering the growth medium or separating one more components from the growth medium. In 506, the growth medium is contacted with a gas including carbon dioxide (e.g., air). In 508, some of the carbon dioxide in the gas is transferred to the growth medium to yield an enriched growth medium. The concentration of dissolved carbon dioxide in the enriched growth medium exceeds the concentration of dissolved carbon dioxide in the growth medium, where dissolved carbon dioxide includes ions formed by the reaction of carbon dioxide with a species in solution, including such ions formed in the growth medium and added to the growth medium. In 510, the enriched growth medium is processed to increase or decrease the concentration of a component in the enriched growth medium. For example, the concentration of dissolved carbon dioxide may be increased, or the concentration of a buffer species may be decreased. In 512, the enriched growth medium is provided to the algaculture facility, thereby increasing the concentration of dissolved carbon dioxide in the bulk growth medium of the algaculture facility and enhancing the growth of algae therein.

In certain cases, one or more operations shown in FIG. 5 may be omitted, one or more additional operations may be added, or both. In some cases, the order of the operations shown in FIG. 5 may be changed, or combinations of operations may be performed.

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

While this specification contains many specific implementation details, these should not be construed as limitations on the scope of any embodiments or of what may be claimed, but rather as descriptions of features specific to particular implementations of particular embodiments. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

A number of embodiments have been described. Nevertheless, it will be understood that various modifications may be made without departing from the spirit and scope of the disclosure. Accordingly, other embodiments are within the scope of the following claims. Further modifications and alternative embodiments of various aspects will be apparent to those skilled in the art in view of this description. Accordingly, this description is to be construed as illustrative only. It is to be understood that the forms shown and described herein are to be taken as examples of embodiments. Elements and materials may be substituted for those illustrated and described herein, parts and processes may be reversed, and certain features may be utilized independently, all as would be apparent to one skilled in the art after having the benefit of this description. Changes may be made in the elements described herein without departing from the spirit and scope of this description as described in the following claims.

1. A method comprising:
contacting a growth medium with a gas comprising carbon dioxide;
transferring some of the carbon dioxide from the gas to the growth medium to yield an enriched growth medium, wherein the concentration of dissolved carbon dioxide in the enriched growth medium exceeds the concentration of dissolved carbon dioxide in the growth medium, and dissolved carbon dioxide comprises ions formed by the reaction of carbon dioxide with a species in solution, including such ions formed in the growth medium and added to the growth medium; and
providing the enriched growth medium to an algaculture facility comprising algae, thereby enhancing growth of the algae in the algaculture facility.

2. The method of claim 1, further comprising removing the growth medium from bulk growth medium in an algaculture facility before contacting the growth medium with the gas.

3. The method of claim 2, wherein removing the growth medium from the algaculture facility, transferring some of the carbon dioxide to the growth medium to yield an enriched growth medium, and providing the enriched growth medium to the algaculture facility is a continuous process.

4. The method of claim 1, further comprising harvesting the algae, a consumer thereof, a metabolic product thereof, or a derivative of the metabolic product from the algaculture facility.

5. The method of claim 1, further comprising subjecting the enriched growth medium to a filtration, dialysis, reverse osmosis, or ion exchange process to alter the concentration of dissolved carbon dioxide in the enriched growth medium before providing the enriched growth medium to the algaculture facility.

6. The method of claim 1, further comprising crystallizing the dissolved carbon dioxide from the enriched growth medium to yield a solid carbon dioxide-containing compound, and providing the solid carbon dioxide-containing compound to the algaculture facility.

7. The method of claim 1, wherein the enriched growth medium comprises a buffer species, and further comprising crystallizing the buffer species from the enriched growth medium to yield a solid buffer species.

8. The method of claim 7, further comprising providing the solid buffer species to the carbon dioxide capture facility.

9. The method of claim 8, wherein providing the solid buffer species to the carbon dioxide capture facility comprises conveying the solid buffer species to the carbon dioxide capture facility.

10. The method of claim 1, further comprising removing dissolved compounds or particulate matter from the growth medium before contacting the growth medium with the gas.

11. The method of claim 10, further comprising providing the dissolved compounds or the particulate matter to the algaculture facility.

12. The method of claim 1, further comprising removing water from the growth medium before contacting the growth medium with the gas.

13. The method of claim 12, further comprising adding water to the enriched growth medium before providing the enriched growth medium to the algaculture facility.

14. The method of claim 1, wherein the growth medium comprises an additive that increases the rate of transfer of the carbon dioxide from the gas to the growth medium.

15. The method of claim 1, further comprising exposing the growth medium to light while contacting the growth medium with the gas.

16. The method of claim 1, wherein the ions formed by the reaction of carbon dioxide with a species in solution and added to the growth medium are added in the form of a solution, a slurry, or a salt.

17. The method of claim 1, wherein the species that react with carbon dioxide in solution comprise water, carbonate, bicarbonate, monobasic phosphate, dibasic phosphate, and tribasic phosphate.

18. A system comprising:
an algaculture facility comprising a growth medium;
a carbon dioxide capture facility coupled to the algaculture facility;
wherein the carbon dioxide capture facility is configured to transfer carbon dioxide from a gas to the portion of the growth medium to yield an enriched growth medium, and
wherein the concentration of dissolved carbon dioxide in the enriched growth medium exceeds the concentration of dissolved carbon dioxide in the growth medium, and dissolved carbon dioxide comprises ions formed by the reaction of carbon dioxide with a species in solution, including such ions formed in the growth medium and added to the growth medium.

19. The system of claim 18, wherein the carbon dioxide capture facility is fluidically coupled to the algaculture facility.

20. The system of claim 18, further comprising a pump configured to transfer the growth medium from the algaculture facility to the carbon dioxide capture facility.

21. The system of claim 18, further comprising a separation unit fluidically interposed between the algaculture facility and the carbon dioxide capture facility, wherein the separation facility is configured to remove one or more components from the growth medium before the growth medium is transferred to the carbon dioxide capture facility, or to remove one or more components from the enriched growth medium before the enriched growth medium is transferred to the algaculture facility.

22. The system of claim 18, further comprising a separation unit fluidically interposed between the algaculture facility and the carbon dioxide capture facility, wherein the separation unit is configured to increase or decrease a concentration of one or more components of the growth medium before the growth medium is transferred to the carbon dioxide capture facility, or to increase or decrease a concentration of one or more components of the enriched growth medium before the enriched growth medium is transferred to the algaculture facility.
